(19) United States
(12) Patent Application Publication
(10) Pub. No.: US 2018/0204046 A1
(43) Pub. Date: Jul. 19, 2018

(54) VISUAL REPRESENTATION LEARNING FOR BRAIN TUMOR CLASSIFICATION

(71) Applicant: Siemens Aktiengesellschaft, Munich (DE)

(72) Inventors: Subhabrata Bhattacharya, Seattle, WA (US); Shanhui Sun, Princeton, NJ (US); Terrence Chen, Princeton, NJ (US); Ali Kamen, Skillman, NJ (US)

(21) Appl. No.: 15/744,887

(22) PCT Filed: Jul. 22, 2016
(86) PCT No.: PCT/US2016/043466

§ 371 Date: Jan. 15, 2018

Related U.S. Application Data
(60) Provisional application No. 62/200,678, filed on Aug. 4, 2015.

(51) Int. Cl. G06K 9/00; G06K 9/46; G06K 9/62; A61B 5/00

(52) U.S. Cl. CPC G06K 9/00147; G06K 9/4609; G06K 9/6255; G06K 2209/05; A61B 5/0042; A61B 5/0084; G06K 9/6262

(57) ABSTRACT

Independent subspace analysis (ISA) is used to learn (42) filter kernels for CLE images in brain tumor classification. Convolution (46) and stacking are used for unsupervised learning (44, 48) with ISA to derive the filter kernels. A classifier is trained (56) to classify CLE brain images based on features extracted using the filter kernels. The resulting filter kernels and trained classifier are used (60, 64) to assist in diagnosis of occurrence of brain tumors during or as part of neurosurgical resection. The classification may assist a physician in detecting whether CLE examined brain tissue is healthy or not and/or a type of tumor.

### RELATED APPLICATIONS

The present patent document claims the benefit of the filing dates under 35 U.S.C. § 119(e) of Provisional U.S. Patent Application Ser. No. 62/200,678, filed Aug. 4, 2015, which is hereby incorporated by reference.

### BACKGROUND

The present embodiments relate to classification of images of brain tumors. Confocal laser endomicroscopy (CLE) is an alternative to in-vivo imaging technology for examining brain tissue for tumors. CLE allows real-time examination of body tissues on a scale that was previously only possible on histological slices. Neurosurgical resection is one of the early adopters of this technology, where the task is to manually identify tumors inside the human brain (e.g., dura matter, occipital cortex, parietal cortex, or other locations) using a probe or endomicroscope. However, this task may be highly time-consuming and error-prone considering the current nascent state of the technology.

Furthermore, with glioblastoma multiforme being an aggressive malignant cerebellar tumor with only 5% survival rates, there has been an increasing demand in employing automatic image recognition techniques for cerebellar tissue classification. Tissues affected by glioblastoma and meningioma are usually characterized by sharp granular and smooth homogeneous patterns, respectively. However, the low resolution of current CLE imaging systems, coupled with the presence of both kind of patterns in healthy tissue in the probing area, makes it extremely challenging for common image classification algorithms to distinguish between types of tumors and/or tumorous and healthy tissue. FIGS. 1A and 1B show CLE image samples taken from cerebellar tissues of different patients diagnosed with glioblastoma multiforme and meningioma, respectively. FIG. 1C shows CLE image samples of healthy cadaveric cerebellar tissues. As seen in FIGS. 1A-C, visual differences under limitations of CLE imagery are not clearly evident as both granular and homogeneous patterns are present in the different images.

Automatic analysis of CLE imagery adapts a generic image classification technique based on bag-of-visual words. Within this technique, images containing different tumors are collected and low-level features (characteristic property of an image patch) are extracted from them as part of a training step. From all images in the training set, representative features also known as visual words are then obtained using vocabulary or dictionary learning, usually either unsupervised clustering or by a supervised dictionary learning technique. After that, each of the collected training images is represented in a unified manner as a bag or collection of visual words in the vocabulary. This is followed by training classifiers, such as support vector machines (SVM) or random forests (RF), to use the unified representation of each image. Given an unlabeled image, features are extracted and the image in turn is represented in terms of already learned visual words. Finally, the representation is input to a pre-trained classifier, which predicts the label of the given image based on its similarity with pre-observed training images. However, the accuracy of classification is less than desired.

### SUMMARY

Systems, methods, and computer readable media are provided for brain tumor classification. Independent subspace analysis (ISA) is used to learn filter kernels for CLE images. Convolution and stacking are used for unsupervised learning with ISA to derive the filter kernels. A classifier is trained to classify CLE images based on features extracted using the filter kernels. The resulting filter kernels and trained classifier are used to assist in diagnosis of occurrence of brain tumors during or as part of neurosurgical resection. The classification may assist a physician in detecting whether CLE examined brain tissue is healthy or not and/or a type of tumor.

In a first aspect, a method is provided for brain tumor classification in a medical image system. Local features are extracted from a confocal laser endomicroscopy image of a brain of a patient. The local feature are extracted using filters learned from independent subspace analysis in each of first and second layers with the second layer based on convolution of output from the first layer with the image. The local features are coded. A machine-learnt classifier classifies from the coded local features. The classification indicates whether the image includes a tumor. An image representing the classification is generated.

In a second aspect, a method is provided for learning brain tumor classification in a medical system. One or more confocal laser endomicroscopes acquire confocal laser endomicroscopy images representing tumorous brain tissue and healthy brain tissue. A machine-learning computer of the medical system performs unsupervised learning on the images in a plurality of layers each with independent subspace analysis. The learning in the layers is performed greedily. A filter filters the images with filter kernels output from the unsupervised learning. In one embodiment, the images as filtered are coded. The outputs of the coding are pooled. In another embodiment, the filtered outputs are pooled without coding. The machine learning computer of the medical system trains, with machine learning, a classifier to distinguish between the images representing the tumorous brain tissue and the images representing the healthy brain tissue based on the pooling of the outputs as an input vector.

In a third aspect, a medical system includes a confocal laser endomicroscope configured to acquire an image of brain tissue of a patient. A filter is configured to convolve the image with a plurality of filter kernels. The filter kernels are machine-learnt kernels from a hierarchy of learnt filter kernels for a first stage, convolution with the learnt filter kernels from the first stage, and the filter kernels learnt from input of results of the convolution. A machine-learnt classifier is configured to classify the image based the convolution of the image with the filter kernels. A display is configured to display results of the classification.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIGS. 1A-C show example CLE images with glioblastoma multiforme, meningioma, and healthy tissue, respectively;

FIG. 2 is a flow chart diagram of one embodiment of a method for learning features with unsupervised learning and training a classifier based on the learnt features;

FIG. 3 illustrates one example of the method of FIG. 2;

FIG. 4 is a table of example input data for CLE-based classifier training;

FIGS. 5 and 6 graphically illustrate example learnt filter kernels associated with different filter kernel sizes;

FIG. 7 is a flow chart diagram of one embodiment of a method for applying a learnt classifier using learnt input features for brain tumor classification of CLE images;

FIGS. 8 and 9 show comparison of results for different classifications; and

FIG. 10 is a block diagram of one embodiment of a medical system for brain tumor classification.

### DETAILED DESCRIPTION OF EMBODIMENTS

Since it is extremely difficult to have a clear understanding of the visual characteristics of tumor-affected regions under the current limitations of CLE imagery, a more efficient data-driven visual representation learning strategy is used. An exhaustive set of filters, which are used to represent even remotely similar images efficiently, are implicitly learned from training data. The learned representation is used as input to any classifier, without any further tuning of parameters.

The quality of a feature or features is important to many image analysis tasks. Useful features may be constructed from raw data using machine learning. The involvement of the machine may better distinguish or identify the useful features as compared to a human. Given the large amount of possible features for images and the variety of sources of images, the machine-learning approach is more robust than manual programming.

A network framework is provided for constructing features from raw image data. Rather than using only pre-programmed features, such as extracted Haar wavelets or local binary patterns (LBP), the network framework is used to learn features for classification. For example, in detection of tumorous brain tissue, local features are learned. Filters enhancing the local features are learned in any number of layers. The output from one layer is convolved with the input image, providing an input for the next layer. Two or more layers are used, such as greedily adding third, fourth or fifth layers were the input of each successive layer is the results from the previous layer. By stacking the unsupervised learning of the different layers with convolution to transition between layers, a hierarchal robust representation of the data effective for recognition tasks is learned. The learning process is performed with the network having any number of layers or depth. At the end, learned filters from one or more layers are used to extract information as an input vector for classification. An optimal visual representation for brain tumor classification is learnt using unsupervised techniques. The classifier is trained, based on the input vector from the learned filters, to classify images of brain tissue.

In one embodiment, surgeons may be assisted by classification of CLE imagery to examine brain tissues on a histological scale in real-time during the surgical resection. The classification of CLE imagery is a difficult problem due to the low signal to noise ratio between tumor inflicted and healthy tissue regions. Moreover, clinical data currently available to train classification algorithms are not annotated cleanly. Thus, off-the-shelf image representation algorithms may not be able to capture crucial information needed for classification purposes. This hypothesis motivates the investigation of unsupervised image representation learning that demonstrate significant success in generic visual recognition problems. A data-driven representation is learnt using unsupervised techniques, which alleviates the necessity of clearly annotated data. For example, an unsupervised algorithm called Independent Subspace Analysis is used in a convolutional neural network framework to enhance robustness of the learned representation. Preliminary experiments show 5-8% improvement over state of the art algorithms on brain tumor classification tasks with negligible sacrifice to computational efficiency.

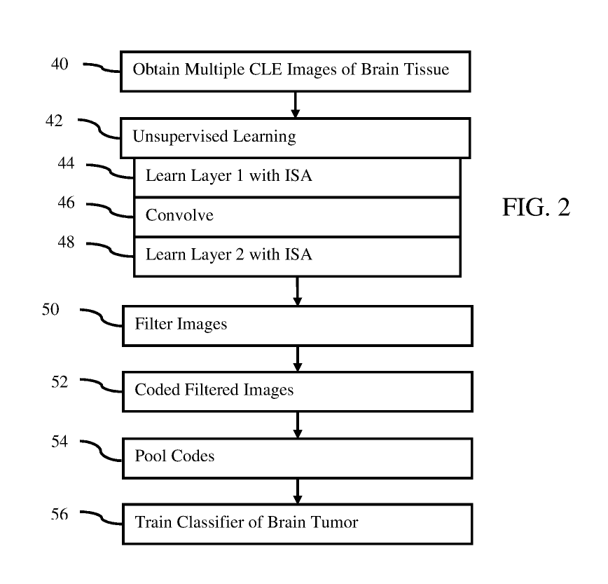

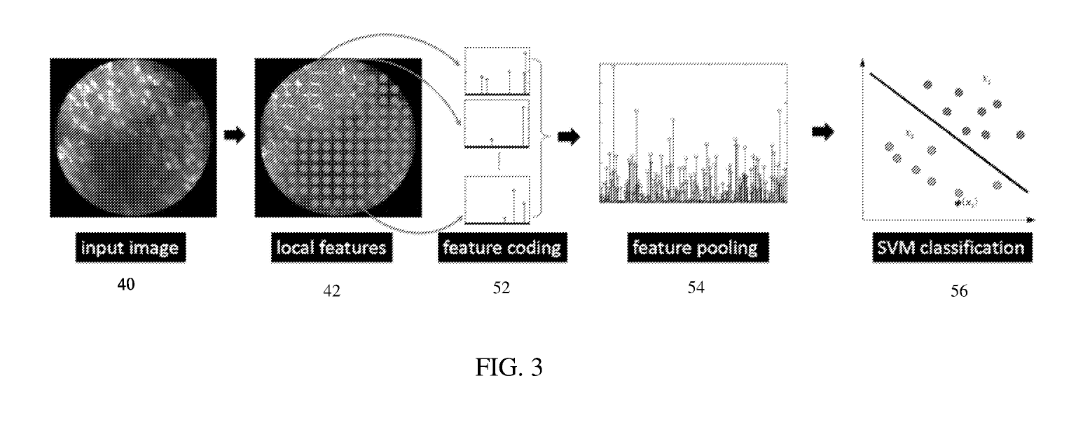

FIG. 2 shows a method for learning brain tumor classification in a medical system. FIG. 3 illustrates an embodiment of the method of FIG. 2. To deal with the similarity of different types of tumors and healthy tissue in CLE imagery, one or more filters are learnt for deriving input vectors to train a classifier. This unsupervised learning of the input vector for classification may allow the classification to better distinguish types of tumors and/or healthy tissue and tumors from each other. A discriminative representation is learnt from the images.

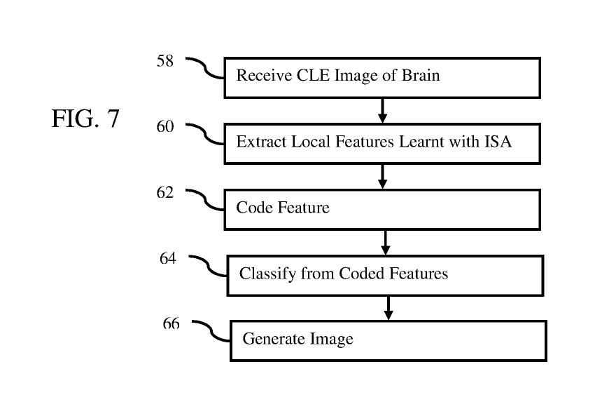

FIGS. 2 and 3 show methods for learning, by a machine in the medical system, a feature or features that distinguish between the states of brain tissue and/or learning a classifier based on the feature or features. The learnt feature or features and/or the trained classifier may be used by the machine to classify (see FIG. 7).

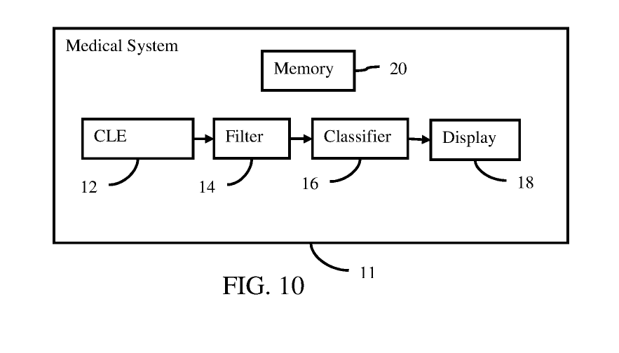

A machine, such as a machine-learning processor, computer, or server, implements some or all of the acts. A CLE probe is used to acquire one or more CLE images. The machine then learns from the CLE images and/or ground truth (annotated tumor or not). The system of FIG. 10 implements the methods in one embodiment. A user may select the image files for training by the processor or to select the image from which to learn features and a classifier by a processor. Use of the machine allows processing large volumes (e.g., images of many pixels and/or many images) of information that may not be efficiently handled by humans, may be unrealistically handled by humans in the needed time frame, or may not even be possible by humans due to subtleties and/or timing.

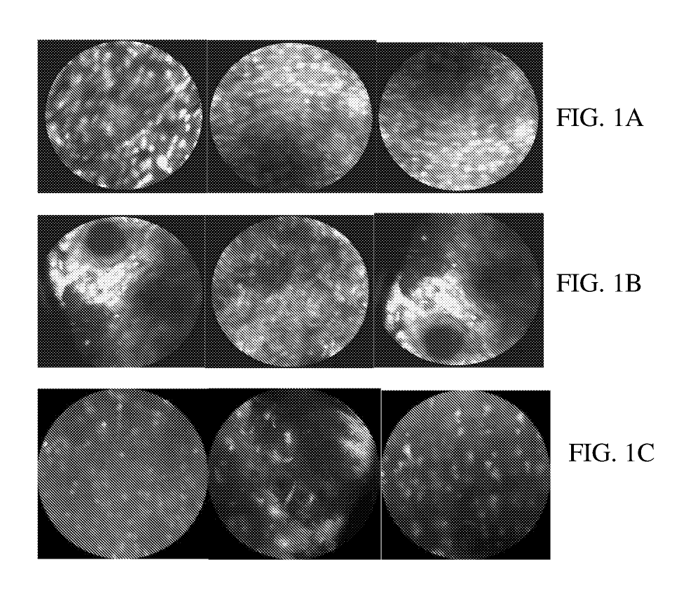

The methods are provided in the orders shown, but other orders may be provided. Additional, different or fewer acts may be provided. For example, acts 44, 46, and/or 48 of FIG. 1 are not provided. As another example, act 56 is not provided. In yet other examples, acts for capturing images and/or acts using detected information are provided. In another embodiment, acts 52 and 54 are not provided. Instead, the classifier is trained using the filtered images or other features extracted from the filtered images. Act 52 may not be performed in other embodiments, such as where the filtered images are pooled without coding.

In act 40, CLE images are acquired. The images are acquired from a database, a plurality of patient records, CLE probes, and/or other sources. The images are loaded from or accessed in a memory. Alternatively or additionally, the images are received over a network interface from any source, such as a CLE probe or picture archiving and communications server (PACS).

The images may be received by scanning a patient and/or from previous scans. The same or different CLE probes are used to acquire the images. The images are from living patients. Alternatively, some or all of the images for training are from cadavers. CLE imaging of the cadavers is performed with the same or different probes. The images are from many different humans and/or many samples of brain tissue imaging. The images represent brain tissue. Different sub-sets of the images represent the brain tissue in different states, such as (1) healthy and tumorous brain tissue and/or (2) different types of tumorous brain tissue.

In one embodiment, a commercially available clinical endo-microscope (e.g., Cellvizio from Mauna Kea Technologies, Paris, France) is used as for CLE imaging. A laser scanning unit, software, a flat panel display and fiber optic probes provide a circular field of view with a diameter of 160 μm, but other structures and/or fields of view may be used. The CLE device is intended for imaging the internal microstructure of tissues in the anatomical tract that are accessed by an endoscope. The system is clinically used during an endoscopic procedure for analysis of sub-surface structures of suspicious lesions, which is referred to as optical biopsy. In a surgical resection application, a neurosurgeon inserts a hand-held probe into a surgical bed (e.g., brain tissue of interest) to examine the remainder of the tumor tissue to be resected. The images acquired during previous resections may be gathered as training data.

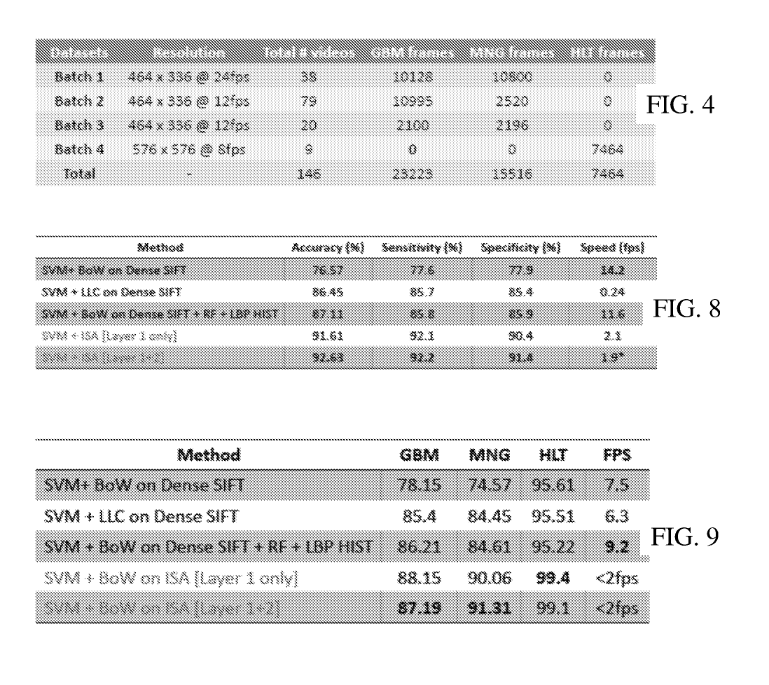

FIG. 4 is a table describing an example collection of CLE images acquired for training. The images are collected in four batches, but other numbers of batches may be used. The first three batches contain video samples that depict occurrences of glioblastoma (GBM) and meningioma (MNG). The last batch has healthy tissue samples collected from a cadaveric head. Other sources and/or types of tumors may be used. For training, the annotations are only available at frame level (i.e., tumor affected regions are not annotated within an image), making it even more difficult for pattern recognition algorithms to leverage on localized discriminative information. Any number of videos is provided for each batch. Any number of image frames of each video may be provided.

Where video is used, some of the images may not contain useful information. Due to the limited imaging capability of CLE devices or intrinsic properties of brain tumor tissues, the resultant images often contain little categorical information and are not useful for recognition algorithms. In one embodiment, to limit the influence of these images, the images are removed. The desired images are selected. Image entropy is used to quantitatively determine the information content of an image. Low-entropy images have less contrast and large runs of pixels with the same or similar values as compared to higher-entropy images. In order to filter uninformative video frames, the entropy of each frame or image is calculated and compared to an entropy threshold. Any threshold may be used. For example, the entropy distribution through a set of data is used. The threshold is selected to leave sufficient (e.g., hundreds or thousands) of images or frames for training. For example, the threshold of 4.05 is used in the dataset of FIG. 4. In alternative embodiments, image or frame reduction is not provided or other approaches are used.

In act 42, a machine-learning computer, processor, or other machine of the medical system performs unsupervised learning on the images. The images are used as inputs to the unsupervised learning to determine features. Rather than or in addition to extracting Haar wavelet or other features, the machine learning determines features specific to the CLE images of brain tissue. A data driven methodology learns image representations that are in turn effective in classification tasks. The feature extraction stage in the computation pipeline (see FIG. 3) encapsulates this act 42.

FIG. 2 shows three acts 44, 46, and 48 for implementing the unsupervised learning of act 42. Additional, different, or fewer acts may be provided, such as including other learning layers and convolutions between the layers. Other non-ISA and/or non-convolution acts may be used.

In the embodiment of FIG. 2, a plurality of layers are trained in acts 44 and 48, with convolution of act 46 being used to relate the stack of layers together. This layer structure learns discriminative representations from the CLE images.

Any unsupervised learning may be used. The learning uses the input, in this case CLE images, without ground truth information (e.g., without the tumor or healthy tissue labels). Instead, the learning highlights contrast or variance common to the images and/or that maximizes differences between the input images. The machine learning is trained by the machine to create filters that emphasize features in the images and/or de-emphasize information of less content.

In one embodiment, the unsupervised learning is independent subspace analysis (ISA) or other form of independent component analysis (ICA). Natural image statistics are extracted by the machine learning from the input images. The natural image statistics learned with ICA or ISA emulate natural vision. Both ICA and ISA may be used to learn receptive fields similar to the V1 area of visual cortex when applied to static images. In contrast to ICA, ISA is capable of learning feature representations that are robust to affine transformation. Other decomposition approaches may be used, such as principle component analysis. Other types of unsupervised learning may be used, such as deep learning.

ICA and ISA may be computationally inefficient when the input training data is too large. Large images of many pixels may result in inefficient computation. The ISA formulation is scaled to support larger input data. Rather than direct ISA to each input image, various patches or smaller (e.g., 16×16 pixels) filter kernels are learnt. A convolutional neural network type of approach uses convolution and stacking. Different filter kernels are learned in act 44 from the input or training images with ISA in one layer. These learned filter kernels are convolved in act 46 with the input or training images. The images are filtered spatially using the filtering kernels windowed to filter each pixel of the images. The filtered images resulting from the convolution are then input to ISA in another layer. Different filter kernels are learned in act 48 from the filtered images resulting from the convolution. The process may repeat or may not repeat with further convolution and learning.

The output patches are filter kernels used for feature extraction in classification. The convolution neural network approach to feature extraction involves learning features with small input filter kernels, which are in turn convolved with a larger region of the input data. The input images are filtered with the learned filter kernels. The outputs of this convolution are used as input to the layer above. This convolution followed by stacking technique facilitates learning a hierarchical robust representation of the data effective for recognition tasks.

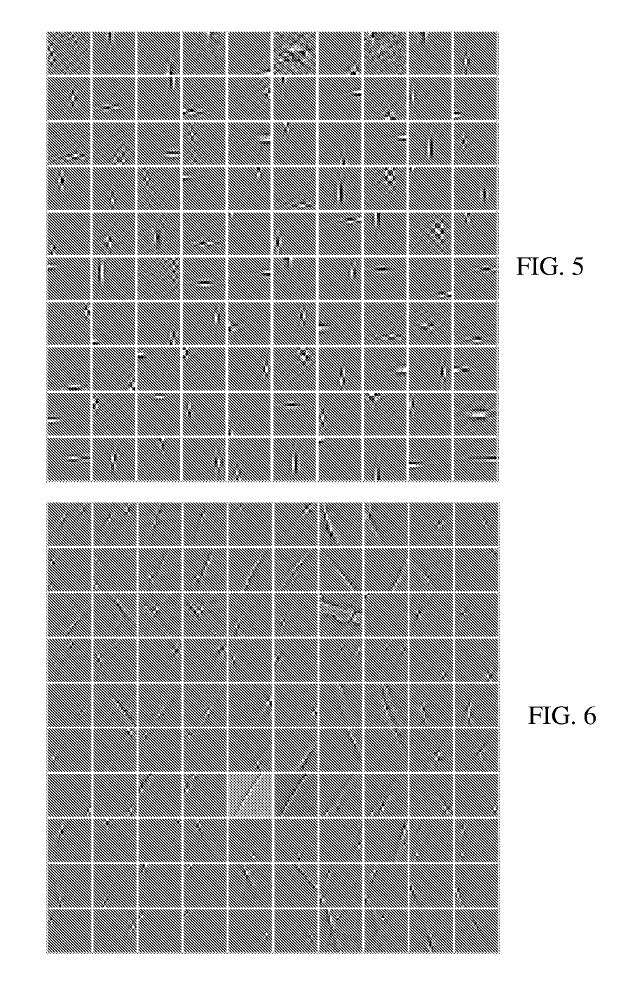

Any number of filter kernels or patches may be created by learning. FIGS. 5 and 6 each show 100 filter kernels, but more or fewer may be provided. The filter kernel size may result in different filter kernels. FIG. 5 shows filter kernels as 16×16 pixels. FIG. 6 shows filter kernels learned using the same input images, but with filter kernel sizes of 20×20 pixels. Greater filter kernel sizes result in greater computational inefficiency. Different filter kernel sizes affect the learning of the discriminative patterns from the images.

For a given layer, ISA learning is applied. Any now know or later developed ISA may be used. In one embodiment, the ISA learning uses a multi-layer network, such as a multi-layer network within one or each of the stacked layers of acts 44 and 48. For example, square and square root non-linearities are used in the learning of the multi-layer network for a given performance of ISA. The square is used in one layer and the square root in another layer of the multi-layer network of the ISA implementation.

In one embodiment, the first layer units are simple units and the second layer units are pooling units. There are k simple units and m pooling units in the multi-layer ISA network. For a vectorized input filter kernel x∈Rⁿ, n being the input dimension (number of pixels in a filter kernel), the weights W∈R^{m×k} in the first layer are learned, while the weights V∈R^{k×n} of the second layer are fixed to represent the subspace structure of the neurons in the first layer. In other words, the first layer is learned, then the second layer. Specifically, each of the second layer hidden units pools over a small neighborhood of adjacent first layer units. The activation of each pooling unit is given by:

pᵢ(x;W,V)=[Σₖ₌₁^{m}Vᵢₖ(Σⱼ₌₁ⁿWₖⱼxⱼ)]^{1/2} (1)

where p is the activation of the second layer output, W are weight parameters of the first layer, V are weights parameters of the second layer, and j and k are indices. The parameters W are learned through finding sparse feature representations in the pooling layer, by solving the following optimization problem over all T input samples:

min_{W}Σₜ₌₁^{T}(Σᵢ₌₁^{m}pᵢ(xₜ;W,V)),s.t.WW^{T}=1 (2)

where T is an index, and the orthonormal constraint WW^{T}=1 ensures the features are diverse. FIGS. 5 and 6 show subsets of features learned after solving the problem in Equation (2) using different input filter kernel dimensions. Other ISA approaches, layer units, non-linearities, and/or multi-layer ISA networks may be used.

For empirical analysis, the filters are learned from different input filter kernel dimensions. However, the standard ISA training algorithm becomes less efficient when input filter kernels are large as for every step of projected gradient descent, there is a computational overhead for an orthogonalization method. This overhead cost grows as a cubic function of the input dimension of the filter kernel size. Using a convolution neural network architecture that progressively makes use of PCA and ISA as sub-units for unsupervised learning may overcome the computational inefficiency, at least in part.

The outputs of one of the layers in the stacking (e.g., output of act 44) may be whitened, such as with principle component analysis (PCA), prior to use in convolution and/or learning in a subsequent layer. First, the ISA algorithm is trained on small input filter kernels. Next, this learned network is convolved with a larger region of the input image. The combined responses of the convolution step are then given as input to the next layer, which is also implemented by another ISA algorithm with PCA as a preprocessing step. The PCA preprocessing is whitening to ensure that the following ISA training step only receives low dimensional inputs.

The learning performed in acts 44 and 48 is performed greedily. A hierarchal representation of the images is learned layer wise, such as done in deep learning. The learning of the first layer in act 44 is performed until convergence before training the second layer in act 48. By greedy training, the training time requirement is reduced to less than only a couple hours on a standard laptop hardware given the data set of FIG. 4.

Once the patches or filter kernels are learned by machine learning using the input training images, a visual recognition system is trained to classify from input features extracted with the filter kernels. The input training images for machine learning the classification are filtered in act 50 with the filter kernels. A filter convolves each training image with each filter kernel or patches output from the unsupervised learning. The filter kernels output by the final layer (e.g., layer 2 of act 48) are used, but filter kernels from the beginning (e.g., layer 1 of act 44) or intermediate layers may be used as well.

For each input training image, a plurality of filtered images is output. The plurality is for the number of filter kernels being used. These filtered images are a visual representation that may be used for better classification than using the images without filtering.

Any visual recognition system may be used, such as directly classifying from the input filtered images. In one embodiment, features are further extracted from the filtered images and used as the input. In the embodiment of FIGS. 2 and 3, the dimensionality or amount of input data is reduced by coding in act 52 and pooling of the codes in act 54.

In act 52, the filtered images are coded. The coding reduces the data used for training the classifier. For example, the filtered images each have thousands of pixels with each pixel being represented by multiple bits. The coding reduces the representation of a given image by half or more, such as providing data with a size of only hundreds of pixels.

Any coding may be used. For example, clustering (e.g., k-means clustering) or PCA is performed on the filtered images. As another example, a vocabulary is learned from the filtered images. The filtered images are then represented using the vocabulary. Other dictionary learning approaches may be used.

In one embodiment, the recognition pipeline codes similar to a Bag-of-Words based method. 10% or other number of descriptors (i.e., filtered images and/or filter kernels to use for filtering) are randomly selected from the training split, and k-means (k=512 is empirically determined from one of the training testing split) clustering is performed to construct four or other number of different vocabularies. Features from each frame are then quantized using these different sets of vocabularies.

In act 54, the processor or computer pools outputs of the coding. The pooling operation computes a statistic value from all encoded local features, e.g., mean value (average pooling) or max value (maximum pooling). This is used to further reduce dimensionality and improve robustness to certain variation, e.g., translation. In the example of K-means based coding, local feature after convolutions is projected to one entry of the K-means based vocabulary. The pooling operation in this embodiment is applied to the same entry of all the local feature, for example, average operation. Pooled features are provided for each of the training images and test images. Pooling may be provided without the coding of act 52.

In act 56, the machine-learning computer of the medical system trains a classifier to distinguish between the images representing the tumorous brain tissue and the images representing the healthy brain tissue and/or between images representing different types of tumors. Machine learning is used train a classifier to distinguish between the content of images. Many examples of each class are provided to statistically relate combinations of input values to each class.

Any type of machine learning may be used. For example, a random forest or support vector machine (SVM) is used. In other examples, a neural network, Bayesian network, or other machine learning is used. The learning is supervised as the training data is annotated with the results or classes. A ground truth from medical experts, past diagnosis, or other source is provided for each image for the training.

The input vector used to train the classifier is the pooled codes. The output of the pooling, coding, and/or filtering is used as an input to the training of the classifier. Other inputs, such as patient age, sex, family history, image features (e.g., Haar wavelet), or other clinical information, may be used in addition to the features extracted from the unsupervised learning. The input vector and the ground truth for each image are used as training data to train the classifier. For example, a support vector machine is trained with a radial basis function (RBF) kernel using parameters chosen using a coarse grid search, such as down sampling the images or coding for further data reduction. The resultant quantized representations from the pooled codes are used to train the SVM classifier with the RBF kernel. A linear kernel is used in alternative embodiments.

The classifier as trained is a matrix. This matrix and the filter kernels or patches are output from the training in FIGS. 2 and 3. These extracted filters and classifier are used in an application to classify for a given patient. FIG. 7 shows one embodiment of a method for brain tumor classification in a medical imaging system. The method uses the learnt patches and the trained classifier to assist in diagnosis of a given patient. The many training examples are used to train so that the classifier may be used to assist diagnosis of other cases.

The same or different medical imaging system used for training is used for application. For a cloud or server based system, the same computer or processor may both learn and apply the learnt filter kernels and classifier. Alternatively, a different computer or processor is used, such as learning with a workstation and applying with a server. For a local based application, a different workstation or computer applies the learnt filter kernels and classifier than the workstation or computer used for training.

The method is performed in the order shown or a different order. Additional, different, or fewer acts may be provided. For example, where the classification is directly trained from the filtered image information without coding, act 62 may not be performed. As another example, the classification is output over a network or stored in memory without generating the image in act 66. In yet another example, acts for scanning with a CLE are provided.

In act 58, one or more CLE images of a brain are acquired with CLE. The image or images are acquired by scanning the patient with CLE, from a network transmission, and/or from memory. In one embodiment, a CLE probe is positioned in a patient's head during a resection. The CLE is performed during surgery. The resulting CLE images are generated.

Any number of CLE images may be received. Where the received CLE image is part of a video, all of the images of the video may be received and used. Alternatively, a sub-set of images is selected for classification. For example, frame entropy is used (e.g., entropy is calculated and a threshold applied) to select a sub-set of one or more images for classification.

In act 60, a filter and/or classifier computer extract local features from the CLE image or images for the patient. The filter filters the CLE image with the previously learned filter kernels, generating a filtered image for each filter kernel. The filters learned from ISA in a stacked (e.g., multiple layers of ISA) and convolution (e.g., convolution of the training images with filters output by one layer to create the input for the next layer) are used to filter the image from a given patient for classification. The sequentially learned filters or patches are created by ISA. The filters or patches of the last layer are output as the filter kernels to be used for feature extraction. These output filter kernels are applied to the CLE image of the patient.

Any number of filter kernels or patches may be used, such as all the learned filter kernels or a fewer number based on determinative filter kernels identified in the training of the classifier. Each filter kernel is centered over each pixel or other sampling of pixels and a new pixel value calculated based on the surrounding pixels as weighted by the kernels.

The output of the filtering is the local features. These local features are filtered images. The filtering enhances some aspects and/or reduces other aspects of the CLE image of the patient. The aspects to enhance and/or reduce, and by how much, was learned in creating the filter kernels.

In act 62, local feature represented in the filtered image are coded. The features are quantified. Using image processing, a classification processor determines values representing the features of the filtered image. Any coding may be used, such as applying principle component analysis, k-means analysis, clustering, or bag-of-words to the filtered images. The same coding used in the training is used for application for the given patient. For example, the learned vocabulary is used to code the filtered images as a bag-of-words. The coding reduces the amount or dimensionality of the data. Rather than having pixel values for each filtered image, the coding reduces the number of values for input to the classifier.

Each filtered image is coded. The codes from all or some of the filtered images created from the CLE image of the patient are pooled. In alternative embodiments, pooling is not used. In yet other embodiments, pooling is provided without coding.

In act 64, a machine-learnt classifier classifies the CLE image from the coded local features. The classifier processor receives the codes or values for the various filtered images. These codes are the input vector for the machine-learnt classifier. Other inputs may be included, such as clinical data for the patient.

The machine-learnt classifier is a matrix or other representation of the statistical relationship of the input vector to class. The previously learnt classifier is used. For example, the machine-learnt classifier is a SVM or random forest classifier learned from the training data.

The classifier outputs a class based on the input vector. The values of the input vector, in combination, indicate membership in the class. The classifier outputs a binary classification (e.g., CLE image is or is not a member—is or is not tumorous), selects between two classes (e.g., healthy or tumorous), or selects between three or more classes (e.g., classifying whether or not the CLE image includes glioblastoma multiforme, meningioma, or healthy tissue). Hierarchal, decision tree, or other classifier arrangements may be used to distinguish between healthy, glioblastoma multiforme and/or meningioma. Other types of tumors and/or other diagnostically useful information about the CLE image may be classified.

The classifier indicates the class for the entire CLE image. Rather than identifying the location of a tumor in the image, the classifier indicates whether the image represents a tumor or not. In alternative embodiments, the classifier or an additional classifier indicates the location of a suspected brain tumor.

In act 66, the classifier processor generates an image representing the classification. The generated image indicates whether the CLE image has a tumor or not or the brain tissue state. For example, the CLE image is output with an annotation, label, or coloring (e.g., tint) indicating the results of the classification. Where the classifier outputs a probability for the results, the probability may be indicated, such as indicating the type of tumor and the percent likelihood estimated for that type of tumor being represented in the CLE image.

The low-level feature representation may be a decisive factor in automatic image recognition tasks or classification. The performance of the ISA based stacking and convolution to derive the feature representation is evaluated against other different feature representation baselines. For each approach, a dense sampling strategy is used during the feature extraction phase to ensure a fair comparison across all feature descriptors. From each CLE image frame, 500 sample point or key points are uniformly sampled after applying a circular region of interest at approximately the same radius as the endoscopic lens.

Each key point is described using the following descriptor types (i.e., the approaches to low-level feature representation): stacked and convolved ISA, scale invariant feature transform (SIFT), and local binary patterns (LBP). These descriptors capture quantized gradient orientations of pixel intensities in a local neighborhood.

A recognition pipeline, similar to the Bag-of-Words (BOW) based method, is implemented for the dense SIFT feature modality as follows: 10% of descriptors are randomly selected from the training split, and k-means (k=512 is empirically determined from one of the training testing split) clustering is performed to construct 4 different vocabularies. Features from each frame are then quantized using these different sets of vocabularies. Locally constrained linear coding (LLC) may be used instead. The resultant quantized representation is used to train an SVM classifier with an RBF kernel. The parameters of the SVM classifier are chosen using a coarse grid search algorithm.

For classification with the LBP features, the LBP histograms are used directly to train a random forest classifier with 8 trees with a maximum depth of 16 levels for each tree. The output confidences from each representation-classifier combinations are then merged using a straightforward multiplicative fusion algorithm. Thus, the decision for a frame is obtained.

In order to make a detailed comparison, SIFT or LBP descriptors are replaced with the feature descriptor learned using the pre-trained two-layered ISA network (i.e., stacked and convolved ISA). The computational pipeline, including vector quantization and classifier training, is conceptually similar to the baseline (SIFT and LBP) approaches.

FIG. 8 shows average accuracy, sensitivity, and specificity as performance metrics for a two class (i.e., binary) classification experiment. Glioblastoma is the positive class, and meningioma is the negative class. This is specifically performed to find how different methods compare in a relatively simpler task as compared to distinguishing between three classes. The accuracy is given by the ratio of all true classifications (positive or negative) against all samples. Sensitivity, on the other hand, is the proportion of positive samples that are detected as positive (e.g., Glioblastoma). Finally, specificity relates to the classification framework's ability to correctly identify negative (e.g., Meningioma) samples. The final column reports the computational speed of all the methods in frames classified per second.

FIG. 9 reports the individual classification accuracy for each of three classes (Glioblastoma (GBM), Meningioma (MNG) and Healthy tissue (HLT)). The speed in frames classified per second is also compared. The convolution operation in the ISA approach is not optimized for speed, but could be through hardware (e.g., parallel processing) and/or software. In all cases, an average of 6% improvement is provided by the ISA approach over the SIFT and LBP approaches.

ISA, with or without the stacking and convolution within the stack, provides a slower but efficient strategy to extract features that enable effective representation learning directly from data without any supervision. Significant performance improvement over state of the art conventional methods (SIFT and LBP) is shown on an extremely challenging task of brain tumor classification from CLE image.

FIG. 10 shows a medical system 11. The medical system 11 includes a confocal laser endomicroscope (CLE) 12, a filter 14, a classifier 16, a display 18, and a memory 20, but additional, different, or fewer components may be provided. For example, a coder is provided for coding outputs of the filter 14 for forming the input vector to the classifier 16. As another example, a patient database is provided for mining or accessing values input to the classifier (e.g., age of patient). In yet another example, the filter 14 and/or classifier 16 are implemented by a classifier computer or processor. In other examples, the classifier 16 is not provided, such as where a machine-learning processor or computer is used for training. Instead, the filter 14 implements convolution and the machine-learning processor performs unsupervised learning of image features (e.g., ISA) and/or training of the classifier 16.

The medical system 11 implements the methods of FIGS. 2, 3, and/or 7. The medical system 11 performs training and/or classifies. The training is to learn filters or other local feature extractors to be used for classification. Alternatively or additionally, the training is of a classifier of CLE images of brain tissue based on input features learned through unsupervised learning. The classifying uses the machine-learnt filters and/or classifier. The same or different medical system 11 is used for training and application (i.e., classifying). Within training, the same or different medical system 11 is used for unsupervised training to learn the filters 14 and for training the classifier 16. Within application, the same or different medical system 11 is used for filtering with the learnt filters and for classification. The example of FIG. 10 is for application. For training, a machine-learning processor is provided to create the filter 14 and/or the classifier 16.

The medical system 11 includes a host computer, control station, workstation, server, or other arrangement. The system includes the display 18, memory 20, and a processor. Additional, different, or fewer components may be provided. The display 18, processor, and memory 20 may be part of a computer, server, or other system for image processing images from the CLE 12. A workstation or control station for the CLE 12 may be used for the rest of the medical system 11. Alternatively, a separate or remote device not part of the CLE 12 is used. Instead, the training and/or application are performed remotely. In one embodiment, the processor and memory 20 are part of a server hosting the training or application for use by the operator of the CLE 12 as the client. The client and server are interconnected by a network, such as an intranet or the Internet. The client may be a computer for the CLE 12, and the server may be provided by a manufacturer, provider, host, or creator of the medical system 11.

The CLE 12 is an endomicroscope for imaging brain tissue. Fluorescence confocal microscopy, multi-photon microscopy, optical coherence tomography, or other types of microscopy may be used. In one embodiment, laser light is used to excite fluorophores in the brain tissue. The confocal principle is used to scan the tissue, such as scanning a laser spot over the tissue and capturing images. A fiber or fiber bundles are used to form the endoscope for the scanning. Other CLE devices may be used.

The CLE 12 is configured to acquire an image of brain tissue of a patient. The CLE 12 is inserted into a head of a patient during brain surgery, and the adjacent tissue is imaged. The CLE 12 may be moved to create a video of the brain tissue.

The CLE 12 outputs the image or images to the filter 14 and/or the memory 20. For training, the CLE 12 or a plurality of CLEs 12 provide images to a processor. For the application example of FIG. 10, the CLE image or images for a given patient are provided to the filter 14 directly or through the memory 20.

The filter 14 is a digital or analog filter. As a digital filter, a graphics processing unit, processor, computer, discrete components, and/or other devices are used to implement the filter 14. While one filter 14 is shown, a bank or plurality of filters 14 may be provided in other embodiments.

The filter 14 is configured to convolve the CLE image from the CLE 12 with each of a plurality of filter kernels. The filter kernels are machine-learnt kernels. Using a hierarchy in the training, filter kernels are learned using ISA for a first stage, the learnt filter kernels are then convolved with the images input to the first stage, and then the filter kernels are learned using ISA in a second stage where the input images are the results of the convolution. In alternative embodiments, other component analysis than ISA is used, such as PCA or ICA. Convolution and stacking are not used in other embodiments.

The result of the unsupervised learning is filter kernels. The filter 14 applies the learnt filter kernels to the CLE image from the CLE 12. At any sampling or resolution, the CLE image is filtered using one of the learned filter kernels. The filtering is repeated or performed in parallel by the filter 14 for each of the filter kernels, resulting in a filtered image for each filter kernel.

The machine-learnt classifier 16 is a processor configured with a matrix from the memory 20. The configuration is the learned relationship of the inputs to the output classes. The previously learned SVM or other classifier 16 is implemented for application.

The classifier 16 is configured to classify the CLE image from the CLE 12 based on the convolution of the image with the filter kernels. The outputs of the filter 14 are used for creating the input vector. A processor or other device may quantify the filtered images, such as applying a dictionary, locality constraint linear coding, PCA, bag-of-words, clustering, or other approach. For example, the processor implementing the classifier 16 codes the filtered images from the filter 14. Other input information may be gathered, such as from the memory 20.

The input information is input as an input vector into the classifier. In response to the input values, the classifier 16 outputs the class of the CLE image. The class may be binary, hierarchal, or multi-class. A probability or probabilities may be output with the class, such as 10% healthy, 85% GBM, and 5% MNG.

The display 18 is a CRT, LCD, projector, plasma, printer, smart phone, or other now known or later developed display device for displaying the results of the classification. The results may be displayed with the CLE image. For example, the display 18 displays the CLE image with an annotation for the class. As another example, tabs or other references to any images classified as not healthy or other label are provided. In response to user selection, the CLE image classified as not healthy for a given tab is displayed. The user may cycle through the tumorous CLE images to confirm the classified diagnosis or to use the classified diagnosis as a second opinion.

The memory 20 is an external storage device, RAM, ROM, database, and/or a local memory (e.g., solid-state drive or hard drive). The memory 20 may be implemented using a database management system (DBMS) managed by the processor and residing on a memory, such as a hard disk, RAM, or removable media. Alternatively, the memory 20 is internal to the processor (e.g. cache).

The outputs of the filtering, the filter kernels, the CLE image, the matrix for the classifier 16, and/or the classification may be stored in the memory 20. Any data used as inputs, results, and/or intermediary processing may be stored in the memory 20.

The instructions for implementing the training or application processes, methods and/or techniques discussed herein are stored in the memory 20. The memory 20 is a non-transitory computer-readable storage media or memories, such as a cache, buffer, RAM, removable media, hard drive or other computer readable storage media. The same or different non-transitory computer readable media may be used for the instructions and other data. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination.

In one embodiment, the instructions are stored on a removable media device for reading by local or remote systems. In other embodiments, the instructions are stored in a remote location for transfer through a computer network. In yet other embodiments, the instructions are stored within a given computer, CPU, GPU or system. Because some of the constituent system components and method steps depicted in the accompanying figures may be implemented in software, the actual connections between the system components (or the process steps) may differ depending upon the manner in which the present embodiments are programmed.

A processor of a computer, server, workstation or other device implements the filter 14 and/or the classifier 16. A program may be uploaded to, and executed by, the processor comprising any suitable architecture. Likewise, processing strategies may include multiprocessing, multitasking, parallel processing and the like. The processor is implemented on a computer platform having hardware, such as one or more central processing units (CPU), a random access memory (RAM), and input/output (I/O) interface(s). The computer platform also includes an operating system and microinstruction code. The various processes and functions described herein may be either part of the microinstruction code or part of the program (or combination thereof) which is executed via the operating system. Alternatively, the processor is one or more processors in a network.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

1. A method for brain tumor classification in a medical image system, the method stored as a computer program on a non-transitory memory that when executed by a processor performs steps comprising:
extracting local features from a confocal laser endomicroscopy image of a brain of a patient, the local feature extracted using filters learned from independent subspace analysis in each of first and second layers with the second layer based on convolution of output from the first layer with the image;
coding the local features;
classifying with a machine-learnt classifier from the coded local features, the classifying indicating whether or not the image includes a tumor; and
generating an image representing the classification.

2. The method of claim 1 wherein extracting comprises generating filtered images and wherein coding comprises performing principle component analysis, k-means analysis, clustering, or bag-of-words to the filtered images.

3. The method of claim 1 wherein classifying comprises classifying with the machine-learnt classifier comprising a support vector machine classifier.

4. The method of claim 1 wherein classifying comprises classifying whether or not the image includes glioblastoma multiforme, meningioma, or glioblastoma multiforme and meningioma.

5. The method of claim 1 wherein generating the image comprises indicating an image having the tumor.

6. The method of claim 1 wherein extracting as learned from independent subspace analysis comprises filtering the image with filter kernels of the filters, the outputs of the filtering being the local features.

7. The method of claim 1 wherein extracting as learned from independent subspace analysis comprises filtering with the filters learned sequentially in the first and second layers, the first layer comprising patches as the output learned with the independent subspace analysis, the patches convolved with the image, and results of the convolution input to the second layer.

8. The method of claim 1 further comprising:
acquiring the image as one of a plurality of confocal laser endomicroscopy images, the one image selected from the plurality based on frame entropy.

9. A method for learning brain tumor classification in a medical system, the method comprising:
acquiring, with one or more confocal laser endomicroscopes, confocal laser endomicroscopy images representing tumorous brain tissue and healthy brain tissue;
performing, by a machine learning computer of the medical system, unsupervised learning on the images in a plurality of layers each with independent subspace analysis, the learning in the layers being performed greedily;
filtering, by a filter, the images with filter kernels output from the unsupervised learning;
coding the images as filtered;
pooling outputs of the coding;
training, by the machine-learning computer of the medical system, with machine learning a classifier to distinguish features of the images representing the tumorous brain tissue and features of the images representing the healthy brain tissue based on the pooling of the outputs as an input vector.

10. The method of claim 9 wherein acquiring comprises acquiring with different ones of the confocal laser endomicroscopes from different patients.

11. The method of claim 9 wherein performing comprises extracting features for the input vector.

12. The method of claim 9 wherein performing comprises learning a hierarchal representation of the images.

13. The method of claim 9 wherein performing comprises learning a plurality of patches from the images with the independent subspace analysis in a first of the layers, convolving the patches with the images, and learning the filter kernels from results of the convolution with the independent subspace analysis.

14. The method of claim 13 wherein learning the filter kernels and the patches with independent subspace analysis each comprises learning with square and square root non-linearities in a multi-layer network.

15. The method of claim 9 further comprising whitening outputs of a first layer of the unsupervised learning with principle component analysis prior to the unsupervised learning in the second layer.

16. The method of claim 9 wherein filtering comprises convolving, and wherein coding comprises applying one of k-means clustering, principle component analysis, or bag of words the images as filtered.

17. The method of claim 9 wherein coding comprises extracting vocabularies and wherein pooling comprises quantifying the images as filtered with the vocabularies.

18. The method of claim 9 wherein training comprises training a support vector machine with a radial basis function kernel using parameters chosen using a coarse grid search.

19. A medical system comprising:
a confocal laser endomicroscope configured to acquire an image of brain tissue of a patient;
a processor that executes a filter configured to perform a convolution of the image with a plurality of filter kernels, the filter kernels comprising machine-learnt kernels from a hierarchy of learnt kernels for a first stage, the filter kernels learnt from input of results of the convolution; and executes a machine-learnt classifier configured to classify the image based on the convolution of the image with the plurality of filter kernels; and
a display configured to display the classification, wherein the classification distinguishes healthy brain tissue and different types of tumorous brain tissue.

20. The medical system of claim 19 wherein the learnt kernels and the filter kernels comprise independent subspace analysis learnt kernels.
